(19) United States
(12) Patent Application Publication
(10) Pub. No.: US 2024/0343676 A1
(43) Pub. Date: Oct. 17, 2024

(54) PROCESS FOR PREPARING DEUTERATED ORGANIC COMPOUNDS

(71) Applicant: MERCK PATENT GMBH, Darmstadt (DE)

(72) Inventors: PHILIPP STOESSEL, FRANKFURT AM MAIN (DE); CHRISTOPH WETZEL, EHINGEN (DONAU) (DE); STEFAN LEHMANN, OTZBERG (DE)

(21) Appl. No.: 18/750,108

(22) Filed: Jun. 21, 2024

Related U.S. Application Data
(63) Continuation of application No. PCT/EP2022/086545, filed on Dec. 19, 2022.

(30) Foreign Application Priority Data
Dec. 21, 2021 (EP) 21216585.6

(51) Int. Cl. C07C 211/61; B01J 23/42; B01J 23/44; B01J 35/00; C07B 59/00; C07C 25/06; C07D 403/10; C07D 405/14

(52) U.S. Cl. CPC C07C 211/61; B01J 23/42; B01J 23/44; B01J 35/19; C07B 59/004; C07C 25/06; C07D 403/10; C07D 405/14; C07B 2200/05; C07C 2523/42; C07C 2523/44

(57) ABSTRACT

A process for preparing a deuterated organic compound includes the following steps:
a) providing at least one heterogeneous metal catalyst, wherein the providing comprises drying of the metal catalyst;
b) preparing a liquid composition comprising at least one organic compound, the at least one heterogeneous metal catalyst, at least one deuterium source, and at least one aliphatic hydrocarbon as solvent, and
c) heating the composition to deuterate the organic compound.

This application is a Continuation under 35 USC § 111(a) of International Patent Application No. PCT/EP2022/086545 filed Dec. 19, 2022, which claims priority to the EP Application No. 21216585.6 filed Dec. 21, 2021. The entire contents of these applications are incorporated herein by reference in their entirety.

The present invention relates to a process for preparing deuterated organic compounds and to deuterated compounds prepared by that process.

Deuterium is an isotope of hydrogen and has a natural occurrence of 0.015%. Deuterated compounds with a high proportion of deuterium are known, and deuterated aromatic compounds are frequently used in studies of the progression of chemical reactions or metabolic conversions.

Deuterated aromatic compounds are used as starting material for pharmaceutical compounds or labels.

Electronic devices containing organic, organometallic and/or polymeric semiconductors are becoming increasingly important, and are being used in many commercial products for reasons of cost and because of their performance. Examples here include organic-based charge transport materials (for example triarylamine-based hole transporters) in photocopiers, organic or polymeric light-emitting diodes (OLEDs or PLEDs) in readout and display devices or organic photoreceptors in photocopiers. Organic solar cells (O-SCs), organic field-effect transistors (O-FETs), organic thin-film transistors (O-TFTs), organic integrated circuits (O-ICs), organic optical amplifiers and organic laser diodes (O-lasers) are at an advanced stage of development and may have great future significance.

Electronic devices in the context of this invention are understood to mean organic electronic devices containing organic semiconductor materials as functional materials. In particular, the electronic devices represent electroluminescent devices such as OLEDs.

The construction of OLEDs in which organic compounds are used as functional materials is known to the person skilled in the art from the prior art. In general, OLEDs are understood to mean electronic devices having one or more layers which comprise organic compounds and emit light on application of a voltage.

In electronic devices, especially OLEDs, there is a great need to improve performance data, especially lifetime, efficiency and operating voltage. For these aspects, it has not been possible to date to find a satisfactory solution.

Electronic devices typically include cathode, anode and at least one functional, preferably emitting, layer. Apart from these layers, they may also comprise further layers, for example in each case one or more hole injection layers, hole transport layers, hole blocker layers, electron transport layers, electron injection layers, exciton blocker layers, electron blocker layers and/or charge generation layers.

A major influence on the performance data of electronic devices is possessed by the hole transport layers and electron transport layers.

It was recently found that the exchange of a hydrogen atom in an organic electronic device increases the lifetime of the organic electronic device. The cause of this could be that the C—D bond is somewhat stronger than a C—H bond and hence particular breakdown reactions are suppressed.

For preparation of deuterated compounds, it is advantageous when the deuteration is not conducted until a late stage in the preparation process, especially in the case of compounds for electronic devices, since deuteration generally constitutes a very costly step.

It is therefore possible to deuterate precursors for compounds for electronic devices, intermediates of such compounds or the compounds for electronic devices themselves.

Typically, undeuterated compounds are treated with deuterated acids such as D₂SO₄ or D₃PO₄ for several hours in order to obtain deuterated compounds.

It is also possible to convert the undeuterated compound in a deuterated solvent in the presence of a Lewis acid such as aluminum trichloride.

Other processes use high temperatures and electrical voltage or radiation.

Other processes again use D₂ gas, D₂O or a deuterated solvent such as C₆D₆ and a metallic catalyst.

However, the conversion is often very slow and unreliable. It is also the case that deuterium sources such as C₆D₆ or d8-toluene are very costly and not readily available.

JP2020070291 describes a process for preparing deuterated compounds in an aliphatic hydrocarbon having more than 6 carbon atoms as solvent, a deuterium source and a metal catalyst. However, an alcohol is also used additionally as additive. The decalin used is also removable only with difficulty. The compounds obtained are difficult to purify.

WO2016073425A2 describes a process of deuterated compounds at high pressure and temperature in D₂O, optionally with solvent.

It is an object of the present invention to provide a process for preparing deuterated compounds with a high conversion rate and high yield, and sparing use of the deuterium source.

The object is achieved by a process for preparing a deuterated organic compound, comprising the following steps:
a) providing at least one heterogeneous metal catalyst, where the providing comprises the drying of the metal catalyst;
b) preparing a liquid composition comprising at least one organic compound, the at least one heterogeneous metal catalyst, at least one deuterium source, and at least one aliphatic hydrocarbon as solvent,
c) heating the composition to deuterate the organic compound.

In a first step, at least one heterogeneous metal catalyst is provided, which comprises the drying of the metal catalyst.

The heterogeneous metal catalyst is preferably selected from the group comprising platinum, palladium, rhodium, ruthenium, nickel, cobalt, oxides thereof and combinations thereof, preferably platinum or palladium and/or oxides thereof.

The metal in at least one metal oxidation catalyst is preferably in the oxidation state of 0 to 2, preferably 0. At least one metal catalyst is preferably in the form of elemental metal and a metal oxide, preferably of elemental metal.

The metal catalyst preferably comprises at least one heterogeneous metal catalyst. The metal in the metal catalyst is preferably in the form of metal, preferably applied on a solid phase which is insoluble in the composition. The solid phase may be any suitable material, for example carbon such as activated carbon or carbon black, silicates, molecular sieve, polymers. The solid phase is stable under the reaction conditions; carbon is a preferred solid phase. Such catalysts are referred to, for example, as Pd/C or Pt/C.

Preferred metal catalysts are platinum, palladium or mixtures of platinum and palladium, especially preferably as metal, more preferably as heterogeneous catalyst.

The at least one metal catalyst is preferably selected from platinum on carbon (Pt/C), palladium on carbon (Pd/C) or a mixture of Pt/C and Pd/C. In the case of a mixture, preference is given to a mixture of 10:1 to 1:2 of Pt/C to Pd/C, preferably 7:1 to 1:1, especially 5:1 to 1:1, measured by weight.

The metal content on the carbon in the metal catalyst is preferably 1% to 10% by weight, especially 3% to 7% by weight, more preferably 5% by weight.

The molar ratio of catalyst to organic compound is preferably 2:1 to 100:1, especially 2:1 to 70:1, preferably 2:1 to 30:1. With a higher amount of catalyst, it is generally the case that a lower level of by-products is formed.

In one embodiment of the invention, the heterogeneous metal catalyst is moist with water prior to drying, where the water content is at least 10% (by the Karl Fischer test).

The heterogeneous metal catalyst is dried. This is preferably done at elevated temperature, especially at 20° C. to 200° C., preferably at 20 to 100° C., more preferably under reduced pressure, especially below 100 mbar. The drying is preferably effected until the water content is below 5% by weight, preferably 2% by weight (by the Karl Fischer test), preferably below 1% by weight.

Particular preference is given to drying at 50° C. to 70° C. at below 50 mbar, especially at 50 to 70° C. at below 30 mbar, most preferably at 55° C. to 75° C. at 1 to 30 mbar. The drying is preferably conducted for at least 24 hours, especially at least 48 hours. Preference is given to drying for between 24 and 96 hours, especially 48 to 96 hours.

The providing is preferably conducted under air or inert gas such as nitrogen or argon. There is no activation with hydrogen or deuterium gas.

It is indeed the case that the metal catalysts are frequently stored under water-moist conditions. It has now been found that, surprisingly, prior drying, especially when D₂O is used as deuterium source, distinctly improves the activity of the catalyst.

The pretreated metal catalyst can be used in the next step without further treatment.

What is meant by deuteration in the context of the invention is that some or all hydrogen atoms are exchanged for deuterium (D) in the course of the conversion. In a deuterated compound, deuterium is present more than 100 times more frequently than according to natural frequency. In the case of percentage figures, the figure relates to the ratio of deuterium to the sum total of protons and deuterium for a particular compound.

In the next step, a liquid composition comprising the organic compound, the heterogeneous catalyst, at least one deuterium source and at least one aliphatic hydrocarbon as solvent is prepared.

For this purpose, the individual constituents are mixed. The organic compound here may be dissolved and/or partly dispersed in the composition.

The organic compound is preferably dissolved in the composition, especially dissolved under the conditions in step c). This means that the organic compound is dissolved in the composition after heating.

The organic compound is preferably an aromatic or heteroaromatic compound, especially a hydrocarbon compound, or an organometallic compound. This is preferably a compound having at least one aromatic or heteroaromatic ring system. The compound is more preferably suitable for use in an electronic device, especially an OLED, or is a precursor of such a compound.

An aromatic ring system in the context of this invention contains 6 to 60 carbon atoms, preferably 6 to 40 carbon atoms, in the ring system. A heteroaromatic ring system in the context of this invention contains 1 to 60 carbon atoms, preferably 1 to 40 carbon atoms, and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for two or more aryl or heteroaryl groups to be joined by a nonaromatic unit (preferably less than 10% of the atoms other than H), for example a carbon, nitrogen or oxygen atom or carbonyl group. These shall likewise be understood to mean systems in which two or more aryl or heteroaryl groups are joined directly to one another, for example biphenyl, terphenyl, bipyridine or phenylpyridine. For example, systems such as fluorene, 9,9′-spirobifluorene, 9,9-diarylfluorene, triarylamine, diaryl ethers, stilbene, etc. shall thus also be regarded as aromatic ring systems in the context of this invention, and likewise systems in which two or more aryl groups are joined, for example, by a linear or cyclic alkyl group or by a silyl group. Preferred aromatic or heteroaromatic ring systems are simple aryl or heteroaryl groups and groups in which two or more aryl or heteroaryl groups are joined directly to one another, for example biphenyl, terphenyl, quaterphenyl or bipyridine, and also fluorene or spirobifluorene. In the case of the bonded aromatic ring systems, preference is given in particular to ring systems having aryl or heteroaryl groups bonded by nitrogen atoms.

An aryl group in the context of this invention contains 6 to 40 carbon atoms; a heteroaryl group in the context of this invention contains 5 to 40 carbon atoms and at least one heteroatom, with the proviso that the sum total of carbon atoms and heteroatoms is at least 5. The heteroatoms are preferably selected from N, O and/or S. An aryl group or heteroaryl group is understood here to mean either a simple aromatic cycle, i.e. benzene, or a simple heteroaromatic cycle, for example pyridine, pyrimidine, thiophene, etc., or a fused (annelated) aryl or heteroaryl group, for example naphthalene, anthracene, phenanthrene, quinoline, isoquinoline, etc. Aromatics joined to one another by a single bond, for example biphenyl, by contrast, are not referred to as an aryl or heteroaryl group but as an aromatic ring system.

An electron-rich heteroaromatic ring system is characterized in that it is a heteroaromatic ring system containing no electron-deficient heteroaryl groups. An electron-deficient heteroaryl group is a six-membered heteroaryl group having at least one nitrogen atom or a five-membered heteroaryl group having at least two heteroatoms, one of which is a nitrogen atom and the other is oxygen, sulfur or a substituted nitrogen atom, where further aryl or heteroaryl groups may also be fused onto these groups in each case. By contrast, electron-rich heteroaryl groups are five-membered heteroaryl groups having exactly one heteroatom selected from oxygen, sulfur and substituted nitrogen, to which may be fused further aryl groups and/or further electron-rich five-membered heteroaryl groups. Thus, examples of electron-rich heteroaryl groups are pyrrole, furan, thiophene, indole, benzofuran, benzothiophene, carbazole, dibenzofuran, dibenzothiophene or indenocarbazole. An electron-rich heteroaryl group is also referred to as an electron-rich heteroaromatic radical.

An electron-deficient heteroaromatic ring system is characterized in that it contains at least one electron-deficient heteroaryl group, and especially preferably no electron-rich heteroaryl groups.

The organic compound may comprise one or more aliphatic hydrocarbyl radicals, i.e. alkyl, alkenyl or alkynyl groups. It may also be substituted by further groups such as F, CN, Cl, Br, I, alkoxy groups or thioalkyl groups. What is important here is that these groups do not react under the reaction conditions.

In the context of the present invention, the term “alkyl group” is used as an umbrella term both for linear or branched alkyl groups and for cyclic alkyl groups. Analogously, the terms “alkenyl group” and “alkynyl group” are used as umbrella terms both for linear or branched alkenyl or alkynyl groups and for cyclic alkenyl or alkynyl groups.

In the context of the present invention, an aliphatic hydrocarbyl radical or an alkyl group or an alkenyl or alkynyl group which may contain 1 to 40 carbon atoms and in which individual nonadjacent CH₂ groups may also be substituted by O, C═O, (C═O) O are preferably understood to mean the following radicals: methyl, ethyl, n-propyl, i-propyl, n-butyl, i-butyl, s-butyl, t-butyl, 2-methylbutyl, n-pentyl, s-pentyl, t-pentyl, 2-pentyl, neopentyl, cyclopentyl, n-hexyl, s-hexyl, t-hexyl, 2-hexyl, 3-hexyl, neohexyl, cyclohexyl, 1-methylcyclopentyl, 2-methylpentyl, n-heptyl, 2-heptyl, 3-heptyl, 4-heptyl, cycloheptyl, 1-methylcyclohexyl, n-octyl, cyclooctyl, 2-ethylhexyl, 1-bicyclo[2.2.2]octyl, 2-bicyclo[2.2.2]octyl, 2-(2,6-dimethyl) octyl, 3-(3,7-dimethyl) octyl, adamantyl, trifluoromethyl, pentafluoroethyl, 2,2,2-trifluoroethyl, 1,1-dimethyl-n-hex-1-yl, 1,1-dimethyl-n-hept-1-yl, 1,1-dimethyl-n-oct-1-yl, 1,1-dimethyl-n-dec-1-yl, 1,1-dimethyl-n-dodec-1-yl, 1,1-dimethyl-n-tetradec-1-yl, 1,1-dimethyl-n-hexadec-1-yl, 1,1-dimethyl-n-octadec-1-yl, 1,1-diethyl-n-hex-1-yl, 1,1-diethyl-n-hept-1-yl, 1,1-diethyl-n-oct-1-yl, 1,1-diethyl-n-dec-1-yl, 1,1-diethyl-n-dodec-1-yl, 1,1-diethyl-n-tetradec-1-yl, 1,1-diethyl-n-hexadec-1-yl, 1,1-diethyl-n-octadec-1-yl, 1-(n-propyl)-cyclohex-1-yl, 1-(n-butyl) cyclohex-1-yl, 1-(n-hexyl)cyclohex-1-yl, 1-(n-octyl)cyclohex-1-yl and 1-(n-decyl)cyclohex-1-yl, ethenyl, propenyl, butenyl, pentenyl, cyclopentenyl, hexenyl, cyclohexenyl, heptenyl, cycloheptenyl, octenyl, cyclooctenyl, cyclooctadienyl, ethynyl, propynyl, butynyl, pentynyl, hexynyl, heptynyl or octynyl. An alkoxy group having 1 to 40 carbon atoms is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy and 2,2,2-trifluoroethoxy. A thioalkyl group having 1 to 40 carbon atoms is understood to mean especially methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio. In general, alkyl, alkoxy or thioalkyl groups according to the present invention may be straight-chain, branched or cyclic, where one or more nonadjacent CH₂ groups may be replaced by the abovementioned groups; in addition, it is also possible for one or more hydrogen atoms to be replaced by D, F, Cl, Br, I, CN or NO₂, preferably F, Cl or CN, more preferably F or CN.

An aromatic or heteroaromatic ring system which has 5-60 aromatic ring atoms, preferably 5-40 aromatic ring atoms, and may also be substituted in each case by the abovementioned radicals or a hydrocarbyl radical and which may be joined to the aromatic or heteroaromatic system via any desired positions is understood to mean especially groups derived from benzene, naphthalene, anthracene, benzanthracene, phenanthrene, pyrene, chrysene, perylene, fluoranthene, naphthacene, pentacene, benzopyrene, biphenyl, biphenylene, terphenyl, triphenylene, fluorene, spirobifluorene, dihydrophenanthrene, dihydropyrene, tetrahydropyrene, cis- or trans-indenofluorene, cis-or trans-indenocarbazole, cis- or trans-indolocarbazole, cis- or trans-monobenzoindenofluorene, cis-or trans-dibenzoindenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, hexaazatriphenylene, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or groups derived from combinations of these systems, which are especially bonded via single bonds and/or nitrogen atoms.

The wording that two or more radicals together may form a ring system, in the context of the present description, should be understood to mean, inter alia, that the two radicals are joined to one another by a chemical bond with formal elimination of two hydrogen atoms. This is illustrated by the following scheme:

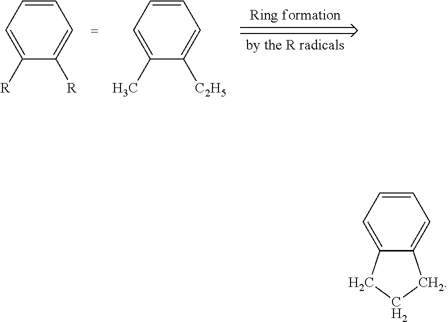

In addition, however, the abovementioned wording shall also be understood to mean that, if one of the two radicals is hydrogen, the second radical binds to the position to which the hydrogen atom was bonded, forming a ring. This will be illustrated by the following scheme:

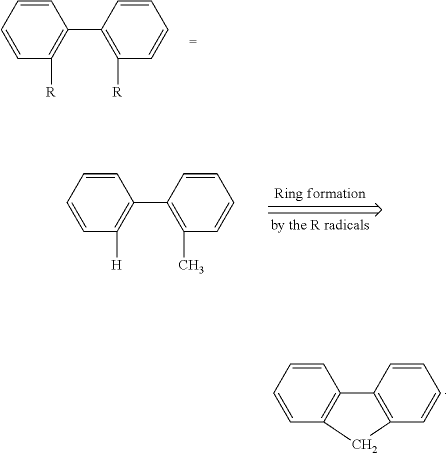

An organometallic compound is preferably a compound comprising copper, molybdenum, tungsten, rhenium, ruthenium, osmium, rhodium, iridium, palladium, platinum, silver, gold or europium, especially compounds such as iridium or platinum, more preferably platinum, which have at least one heteroaromatic ring system. Preference is given to compounds that are suitable as phosphorescent compounds (=triplet emitters). Examples of such compounds can be found in the cited applications for phosphorescent compounds.

These compounds are preferably metal chelate complexes, especially having at least one heteroaromatic ring system as chelate ligand for the metal. Preference is given to a heteroaromatic ring system that binds to the metal via at least one nitrogen atom and via at least one carbon atom. These atoms are preferably each part of an aryl group or heteroaryl group that are bonded via a single bond. Examples of such a compound are 2-phenylpyridine or analogous compounds in which aforementioned aryl groups or heteroaryl groups are joined via a single bond.

The deuterium source is preferably selected from heavy water, D₂O, d6-benzene or d8-toluene, preferably heavy water or D₂O, more preferably D₂O. Heavy water or D₂O in particular are a cheaper deuterium source than the other compounds.

Heavy water means water in which 50 mol % of all hydrogen atoms have been exchanged for deuterium, preferably at least 70 mol %, more preferably at least 80 mol %, in particular at least 90% or 99%.

The solvent serves here to increase the solubility of the organic compound in the composition.

It has been found that, surprisingly, the solvent, especially cycloalkanes, promotes deuteration.

The aliphatic solvent is preferably an aliphatic solvent having a boiling point exceeding 75° C., especially exceeding 80° C. (measured at standard pressure). The solvent is preferably a cycloalkane, preferably a solvent having at least one ring having 6 aliphatic carbon atoms. More preferably cyclohexane, methylcyclohexane or fused cycloalkanes such as decalin. Preference is given to cyclohexane and decalin, especially cyclohexane. The decalin may take the form of the cis or trans isomer or of an isomer mixture.

The solvent is preferably not deuterated. It is preferable that the deuterium source, especially D₂O, is the only deuterated compound in the composition.

The composition preferably does not comprise any aliphatic alcohols, especially having 1 to 10 carbon atoms; the composition preferably does not comprise any aliphatic alcohols. The composition preferably does not comprise any organic compounds having hydroxyl groups.

It is known from the prior art that deuteration is favored in the presence of aliphatic alcohols having 1 to 10 carbon atoms. It has been found that, surprisingly, reaction with the pretreated metal catalyst in the presence of an aliphatic alcohol leads to an increase in breakdown products. In the absence of an aliphatic alcohol, an accelerated and clean deuteration is achieved.

In the composition, the ratio of hydrogen atoms in the organic compound to deuterium in the deuterium source is at least 1:1.5, preferably 1:1.5 to 1:1000, preferably 1:2 to 1:500, more preferably 1:5 to 1:200. Particular preference is given to a ratio of 1:5 to 1:100.

The aliphatic solvent is used in such an amount that the organic compound dissolves at least partly, measured by volume preferably in a weight ratio of deuterium source: solvent of 2:1 to 50:1, preferably 1:1 to 1:30, especially 1:1.5 to 1:30, very particularly 1:1.5 to 1:10. The ideal amount depends on the solubility of the compound.

In step c), the composition is heated, resulting in deuteration.

The reaction may be conducted with equalization of pressure with the environment, i.e. in an open or closed vessel. In the latter case, the autogenous pressure can result in an increase in pressure through the heating. A procedure with equalization of pressure may also mean heating under reflux conditions. Step c) is therefore preferably conducted at a pressure of 1 bar or more. Preferably below 6 bar.

The reaction is preferably not conducted in the presence of additional reactive gases such as H₂ or D₂. The reaction is preferably conducted in an inert atmosphere such as nitrogen or argon. What is meant by “inert” is that the gas or the gas mixture does not react under the process conditions.

It may be necessary to degas the composition prior to the procedure. This can be accomplished, for example, by repeated charging with the reaction atmosphere.

In step c), the reaction is conducted while heating. The heating can be effected at a temperature of at least 40° C., in particular at least 70° C., in particular at least 100° C. The temperature is preferably up to 250° C., especially up to 160° C. More preferably 70° C. to 200° C., especially 70° C. to 160° C. The reaction is preferably not conducted under supercritical conditions.

Depending on the reaction regime, the reaction can also be conducted under reflux. The solvent can then be chosen appropriately, such that the desired reaction temperature is attained.

The inventive process is preferably conducted until deuteration of at least 20%, especially 30%, is attained. This figure is based on the degree of deuteration of the highest mass peak of the product mixture.

As well as deuteration, the formation of by-products in the process is also of high significance. Specifically in the case of application to complex compounds, a lower degree of formation of by-products may be more advantageous than maximum deuteration. Another reason for this is that not all protons of a compound are equally accessible.

Preference is given to conducting the process until a conversion of at least 90% (measured by HPLC) is attained. This means that a maximum of 10% reactant is still present. Preference is given to a conversion of at least 95%.

The reaction is preferably conducted for 1 to 200 hours, especially for 10 to 100 hours.

More preferably, the reaction is conducted until a level of deuteration of at least 20% is achieved with less than 15% by-products, preferably of at least 30% with less than 10% by-products, especially at least 40% with less than 10%.

After cooling, and optional equalization of pressure, the deuterated compound is preferably isolated by known techniques. This may comprise extraction, precipitation, filtration, distillation, chromatography or similar methods.

In one embodiment of the invention, the composition comprises at least one additive for improving deuteration and/or reducing the level of by-products. The at least one additive is preferably selected from alkylamines, preferably alkylamines having alkyl groups having 1 to 40 carbon atoms, where individual nonadjacent CH₂ groups may be substituted by O and at least two alkyl groups together may form a ring, metal salts and/or metal oxides selected from salts or oxides of palladium, platinum, rhodium, ruthenium, silver, gold, copper, nickel or cobalt, preference being given to salts or oxides of silver or palladium, especially of Pd(II). The salts may be, for example, the chlorides, bromides, iodides, nitrates, sulfates, carboxylates such as acetates, propionates, pivalates, for example Pd(OAc)₂, Ag(OAc) or Pd(OPiv)₂. Particular preference is given to carboxylates such as Pd(OAc)₂, Ag(OAc) or Pd(OPiv)₂.

Preferred alkylamines are alkylamines having at least two, preferably three, alkyl groups, especially having 1 to 40 carbon atoms, where individual nonadjacent CH₂ groups may be replaced by O and at least two alkyl groups together may form a ring. Preferred alkyl groups are methyl, ethyl, n-propyl, i-propyl, n-butyl, i-butyl, s-butyl, t-butyl, 2-methylbutyl, n-pentyl, s-pentyl, t-pentyl, 2-pentyl, neopentyl, cyclopentyl, n-hexyl, s-hexyl, t-hexyl, 2-hexyl, 3-hexyl, neohexyl, cyclohexyl, 1-methylcyclopentyl, 2-methylpentyl, n-heptyl, 2-heptyl, 3-heptyl, 4-heptyl, cycloheptyl, 1-methylcyclohexyl. Preference is given to alkylamines having three alkyl groups (tertiary amines) having 1 to 5 carbon atoms, and alkylamines having three alkyl groups where two alkyl groups form a ring, where the ring may contain an oxygen atom.

Examples of such amines are triethylamine, dimethylethylamine, diethylmethylamine, diisopropylethylamine, preference being given to triethylamine. Examples of cyclic amines are morpholine derivatives, especially N-alkylmorpholines such as N-methylmorpholine, N-ethylmorpholine, N-propylmorpholine.

The amine used is preferably soluble in the composition.

Particular preference is given to the aforementioned metal salts.

It has been found that, surprisingly, alkylamines, silver salts and/or palladium salts in particular promote deuteration and reduce the formation of by-products. In this way, it may be possible to conduct the reaction for longer or at higher temperature. The use of the additives may be dependent on the compound to be deuterated.

The additives may be used in different amounts depending on the reaction regime and the organic compound. The at least one additive is preferably used in a molar ratio of additive to organic compound of 1:2 to 1:100, preferably 1:2 to 1:50, especially 1:2 to 1:30.

In a preferred embodiment, the composition comprises at least one aromatic or heteroaromatic compound, platinum on carbon and/or palladium on carbon, D₂O, and cyclohexane and/or decalin, preferably cyclohexane, and optionally at least one additive, where the additive is selected from alkylamines, metal salts and/or metal oxides selected from salts or oxides of palladium, platinum, rhodium, ruthenium, silver, gold, copper, nickel or cobalt.

In a preferred embodiment, the composition consists of at least one aromatic or heteroaromatic compound, platinum on carbon and/or palladium on carbon, D₂O, and cyclohexane and/or decalin, preferably cyclohexane, and optionally at least one additive, where the additive is selected from alkylamines, metal salts and/or metal oxides selected from salts or oxides of palladium, platinum, rhodium, ruthenium, silver, gold, copper, nickel or cobalt.

The compounds deuterated in accordance with the invention are suitable for use in an electronic device, especially in an organic electroluminescent device (OLED). Depending on the substitution, the compounds may be used in different functions and layers.

An electronic device in the context of the present invention is a device comprising at least one layer comprising at least one organic compound. This component may also comprise inorganic materials or else layers formed entirely from inorganic materials.

The electronic device is preferably selected from the group consisting of organic electroluminescent devices (OLEDs), organic integrated circuits (O-ICs), organic field-effect transistors (O-FETs), organic thin-film transistors (O-TFTs), organic light-emitting transistors (O-LETs), organic solar cells (O-SCs), dye-sensitized organic solar cells (DSSCs), organic optical detectors, organic photoreceptors, organic field-quench devices (O-FQDs), light-emitting electrochemical cells (LECs), organic laser diodes (O-lasers) and organic plasmon emitting devices, but preferably organic electroluminescent devices (OLEDs).

The device is more preferably an organic electroluminescent device, comprising anode, cathode and at least one emitting layer, where at least one organic layer that may be an emitting layer, hole transport layer, electron transport layer, hole blocker layer, electron blocker layer or other functional layer comprises at least one compound deuterated in accordance with the invention. The layer is dependent on the substitution of the compound.

Apart from these layers, the organic electroluminescent device may comprise still further layers, for example in each case one or more hole injection layers, hole transport layers, hole blocker layers, electron transport layers, electron injection layers, exciton blocker layers, electron blocker layers, charge generation layers and/or organic or inorganic p/n junctions. It is likewise possible for interlayers having an exciton-blocking function, for example, to be introduced between two emitting layers. However, it should be pointed out that not necessarily every one of these layers need be present.

In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are systems having three emitting layers, where the three layers show blue, green and orange or red emission (the basic construction is described, for example, in WO 2005/011013). The organic electroluminescent device of the invention may also be a tandem OLED, especially for white-emitting OLEDs.

The organic electroluminescent device may comprise one or more phosphorescent emitters.

In this case, the organic electroluminescent device may contain an emitting layer, or it may contain a plurality of emitting layers, where at least one layer contains at least one deuterated compound. In addition, the compound deuterated in accordance with the invention can also be used in an electron transport layer and/or in a hole blocker layer and/or in a hole transport layer and/or in an exciton blocker layer.

The expression “phosphorescent compound” typically refers to compounds where light is emitted through a spin-forbidden transition, for example a transition from an excited triplet state or a state having a higher spin quantum number, for example a quintet state.

Suitable phosphorescent compounds (=triplet emitters) are especially compounds which, when suitably excited, emit light, preferably in the visible region, and also contain at least one atom of atomic number greater than 20, preferably greater than 38, and less than 84, more preferably greater than 56 and less than 80. Preferred phosphorescent compounds are all luminescent complexes with transition metals or lanthanides, especially when they contain copper, molybdenum, tungsten, rhenium, ruthenium, osmium, rhodium, iridium, palladium, platinum, silver, gold or europium, especially compounds containing iridium, platinum or copper. In the context of the present invention, all luminescent iridium, platinum or copper complexes are considered to be phosphorescent emitting compounds.

Examples of the emitters described above can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439, WO 2018/011186, WO 2018/041769, WO 2019/020538, WO 2018/178001, WO 2019/115423 and WO 2019/158453. In general, all phosphorescent complexes as used for phosphorescent OLEDs according to the prior art and as known to those skilled in the art in the field of organic electroluminescence are suitable, and the person skilled in the art will be able to use further phosphorescent complexes without exercising inventive skill. It has been found that, surprisingly, such compounds can be deuterated by the process of the invention.

If the deuterated compound is used as hole transport material in a hole transport layer, a hole injection layer or an electron blocker layer, the compound may be used as pure material, i.e. in a proportion of 100%, in the hole transport layer, or it may be used in combination with one or more further compounds. In a preferred embodiment, the organic layer containing the deuterated compound then additionally contains one or more p-dopants. p-Dopants that are used according to the present invention are preferably those organic electron acceptor compounds that are capable of oxidizing one or more of the other compounds in the mixture.

Particularly preferred embodiments of p-dopants are the compounds disclosed in WO 2011/073149, EP 1968131, EP 2276085, EP 2213662, EP 1722602, EP 2045848, DE 102007031220, U.S. Pat. Nos. 8,044,390, 8,057,712, WO 2009/003455, WO 2010/094378, WO 2011/120709, US 2010/0096600, WO 2012/095143 and DE 102012209523.

Particularly preferred p-dopants are quinodimethane compounds, azaindenofluorenediones, azaphenylenes, azatriphenylenes, I₂, metal halides, preferably transition metal halides, metal oxides, preferably metal oxides containing at least one transition metal or a metal of main group 3, and transition metal complexes, preferably complexes of Cu, Co, Ni, Pd and Pt with ligands containing at least one oxygen atom as binding site. Preference is further given to using transition metal oxides as dopants, preferably oxides of rhenium, molybdenum and tungsten, more preferably Re₂O₇, MoO₃, WO₃ and ReO₃.

The p-dopants are preferably distributed essentially homogeneously in the p-doped layers. This can be achieved, for example, by coevaporation of the p-dopant and the hole transport material matrix.

The deuterated compound can also be used in an emitting layer as matrix material in combination with one or more emitting compounds, preferably phosphorescent compounds.

The proportion of the matrix material in the emitting layer in this case is between 50.0% and 99.9% by volume, preferably between 80.0% and 99.5% by volume, more preferably between 92.0% and 99.5% by volume, for fluorescent emitting layers and between 85.0% and 97.0% by volume for phosphorescent emitting layers.

Correspondingly, the proportion of the emitting compound is between 0.1% and 50.0% by volume, preferably between 0.5% and 20.0% by volume, more preferably between 0.5% and 8.0% by volume, for fluorescent emitting layers and between 3.0% and 15.0% by volume for phosphorescent emitting layers.

An emitting layer of an organic electroluminescent device may also comprise systems containing a multitude of matrix materials (mixed matrix systems) and/or a multitude of emitting compounds. In that case too, in general, the emitting compounds are those which have the smaller proportion in the system, and the matrix materials those which have the greater proportion in the system. In individual cases, however, the proportion of a single matrix material in the system may be smaller than the proportion of a single emitting compound.

Preferred fluorescent emitting compounds are selected from the class of the arylamines. An arylamine or an aromatic amine in the context of the present invention is understood to mean a compound containing three substituted or unsubstituted aromatic or heteroaromatic ring systems bonded directly to the nitrogen. Preferably, at least one of these aromatic or heteroaromatic ring systems is a fused ring system, more preferably having at least 14 aromatic ring atoms. Preferred examples of these are aromatic anthraceneamines, aromatic anthracenediamines, aromatic pyreneamines, aromatic pyrenediamines, aromatic chryseneamines or aromatic chrysenediamines. An aromatic anthraceneamine is understood to mean a compound in which a diarylamino group is bonded directly to an anthracene group, preferably in the 9 position. An aromatic anthracenediamine is understood to mean a compound in which two diarylamino groups are bonded directly to an anthracene group, preferably in the 9,10 positions. Aromatic pyreneamines, pyrenediamines, chryseneamines and chrysenediamines are defined analogously, in which the diarylamino groups are preferably bonded to the pyrene in the 1 position or 1,6 positions. Further preferred emitting compounds are indenofluoreneamines or fluorenediamines, for example according to WO 2006/108497 or WO 2006/122630, benzoindenofluoreneamines or -fluorenediamines, for example according to WO 2008/006449, and dibenzoindenofluoreneamines or -diamines, for example according to WO 2007/140847, and the indenofluorene derivatives having fused aryl groups disclosed in WO 2010/012328. Likewise preferred are the pyrenearylamines disclosed in WO 2012/048780 and in WO 2013/185871. Likewise preferred are the benzoindenofluoreneamines disclosed in WO 2014/037077, the benzofluoreneamines disclosed in WO 2014/106522, the extended benzoindenofluorenes disclosed in WO 2014/111269 and in WO 2017/036574, the phenoxazines disclosed in WO 2017/028940 and in WO 2017/028941, and the fluorine derivatives bonded to furan units or to thiophene units that are disclosed in WO 2016/150544.

Useful matrix materials, preferably for fluorescent compounds, include materials from different substance classes. Preferred matrix materials are selected from the classes of the oligoaryls (e.g. 2,2′,7,7′-tetraphenylspirobifluorene according to EP 676461 or dinaphthylanthracene), especially of the oligoaryls having fused aromatic groups, the oligoarylenevinylenes (e.g. DPVBi or spiro-DPVBi according to EP 676461), the polypodal metal complexes (for example according to WO 2004/081017), the hole-conducting compounds (for example according to WO 2004/058911), the electron-conducting compounds, especially ketones, phosphine oxides, sulfoxides, etc. (for example according to WO 2005/084081 and WO 2005/084082), the atropisomers (for example according to WO 2006/048268), the boronic acid derivatives (for example according to WO 2006/117052) or the benzanthracenes (for example according to WO 2008/145239). Particularly preferred matrix materials are selected from the classes of the oligoarylenes with naphthalene, anthracene, benzanthracene and/or pyrene or atropisomers of these compounds, the oligoarylenevinylenes, the ketones, the phosphine oxides and the sulfoxides. Very particularly preferred matrix materials are selected from the classes of the oligoarylenes comprising anthracene, benzanthracene, benzophenanthrene and/or pyrene or atropisomers of these compounds. An oligoaryl in the context of the present invention is understood to mean a compound in which at least three aryl or arylene groups are bonded to one another. Further preferred are the anthracene derivatives disclosed in WO 2006/097208, WO 2006/131192, WO 2007/065550, WO 2007/110129, WO 2007/065678, WO 2008/145239, WO 2009/100925, WO 2011/054442 and EP 1553154, the pyrene compounds disclosed in EP 1749809, EP 1905754 and US 2012/0187826, the benzanthracenylanthracene compounds disclosed in WO 2015/158409, the indenobenzofurans disclosed in WO 2017/025165, and the phenanthrylanthracenes disclosed in WO 2017/036573.

Preferred matrix materials for phosphorescent compounds are aromatic ketones, aromatic phosphine oxides or aromatic sulfoxides or sulfones, for example according to WO 2004/013080, WO 2004/093207, WO 2006/005627 or WO 2010/006680, triarylamines, carbazole derivatives, e.g. CBP (N,N-biscarbazolylbiphenyl) or according to WO 2005/039246, US 2005/0069729, JP 2004/288381, EP 1205527, WO 2008/086851 or WO 2013/041176, indolocarbazole derivatives, for example according to WO 2007/063754 or WO 2008/056746, indenocarbazole derivatives, for example according to WO 2010/136109, WO 2011/000455, WO 2013/041176 or WO 2013/056776, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2007/063754, WO 2008/056746, WO 2010/015306, WO 2011/057706, WO 2011/060859 or WO 2011/060877, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to WO 2011/042107, WO 2011/060867, WO 2011/088877 and WO 2012/143080, triphenylene derivatives, for example according to WO 2012/048781, lactams, for example according to WO 2011/116865 or WO 2011/137951, or dibenzofuran derivatives, for example according to WO 2015/169412, WO 2016/015810, WO 2016/023608, WO 2017/148564 or WO 2017/148565. It is likewise possible for a further phosphorescent emitter having shorter-wavelength emission than the actual emitter to be present as co-host in the mixture, or a compound not involved in charge transport to a significant extent, if at all, as described, for example, in WO 2010/108579.

Suitable charge transport materials as usable in the hole injection layer or hole transport layer or in the electron blocker layer or in the electron transport layer of the electronic component are, as well as the deuterated compounds, for example, those mentioned in Y. Shirota et al., Chem. Rev. 2007, 107 (4), 953-1010, or other materials as used in these layers according to the prior art.

The one OLED preferably comprises two or more different hole-transporting layers. The deuterated compound may be used in one or more or in all hole-transporting layers. Further compounds which are preferably used in hole-transporting layers of the OLEDs are especially indenofluoreneamine derivatives (for example according to WO 06/122630 or WO 06/100896), the amine derivatives disclosed in EP 1661888, hexaazatriphenylene derivatives (for example according to WO 01/049806), amine derivatives with fused aromatics (for example according to U.S. Pat. No. 5,061,569), the amine derivatives disclosed in WO 95/09147, monobenzoindenofluoreneamines (for example according to

WO 08/006449), dibenzoindenofluoreneamines (for example according to WO 07/140847), spirobifluoreneamines (for example according to WO 2012/034627 or WO 2013/120577), fluoreneamines (for example according to WO 2014/015937, WO 2014/015938, WO 2014/015935 and WO 2015/082056), spirodibenzopyranamines (for example according to WO 2013/083216), dihydroacridine derivatives (for example according to WO 2012/150001), spirodibenzofurans and spirodibenzothiophenes (for example according to WO 2015/022051, WO 2016/102048 and WO 2016/131521), phenanthrenediarylamines (for example according to WO 2015/131976), spirotribenzotropolones (for example according to WO 2016/087017), spirobifluorenes with meta-phenyldiamine groups (for example according to WO 2016/078738), spirobisacridines (for example according to WO 2015/158411), xanthenediarylamines (for example according to WO 2014/072017), and 9,10-dihydroanthracene spiro compounds with diarylamino groups according to WO 2015/086108.

Very particular preference is given to the use of spirobifluorenes substituted in the 4 position by diarylamino groups as hole-transporting compounds, especially to the use of those compounds that are claimed and disclosed in WO 2013/120577, and to the use of spirobifluorenes substituted in the 2 position by diarylamino groups as hole-transporting compounds, especially to the use of those compounds that are claimed and disclosed in WO 2012/034627.

Materials used for the electron transport layer may be any materials that are used as electron transport materials in the electron transport layer according to the prior art. Particularly suitable are aluminum complexes, e.g. Alq3, zirconium complexes, e.g. Zrq4, lithium complexes, e.g. Liq, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives. Further suitable materials are derivatives of the aforementioned compounds as disclosed in JP 2000/053957, WO 2003/060956, WO 2004/028217, WO 2004/080975 and WO 2010/072300.

Preferred cathodes of the electronic component are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally suitable are alloys of an alkali metal or alkaline earth metal and silver, for example an alloy of magnesium and silver. In the case of multilayer structures, in addition to the metals mentioned, it is also possible to use further metals having a relatively high work function, for example Ag or Al, where combinations of the metals such as Ca/Ag, Mg/Ag or Ba/Ag, for example, are generally used. It may also be advantageous to introduce a thin interlayer of a material having a high dielectric constant between a metallic cathode and the organic semiconductor. Examples of suitable materials are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Preferred anodes are materials having a high work function. The anode preferably has a work function of more than 4.5 eV versus vacuum. Firstly suitable for this purpose are metals having a high redox potential, e.g. Ag, Pt or Au. Secondly preferred may also be metal/metal oxide electrodes (e.g. Al/Ni/NiOx, Al/PtOx). For some applications, at least one of the electrodes has to be transparent or partly transparent in order to enable the irradiation of the organic material (organic solar cell) or the emission of light (OLED, O-laser). Preferred anode materials here are conductive mixed metal oxides. Particular preference is given to indium tin oxide (ITO) or indium zinc oxide (IZO). Further preferred are conductively doped organic materials, especially conductively doped polymers. In addition, the anode may also consist of two or more layers, for example of an inner layer of ITO and an outer layer of a metal oxide, preferably tungsten oxide, molybdenum oxide or vanadium oxide.

The device is structured appropriately (according to the application), contact-connected and finally sealed, in order to rule out harmful effects of water and air.

In the further layers of the organic electroluminescent device, it is possible to use any materials as typically used according to the prior art. The person skilled in the art will therefore be able, without exercising inventive skill, to use all the materials known for organic electroluminescent devices in combination with the deuterated compounds. It is also possible to deuterate the aforementioned compounds, especially the aromatic or heteroaromatic compounds, by the process of the invention, especially in order to improve their lifetime.

Additionally preferred is an organic electroluminescent device, characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. However, it is also possible that the initial pressure is even lower, for example less than 10⁻⁷ mbar.

Preference is likewise given to an organic electroluminescent device, characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured.

Preference is additionally given to an organic electroluminescent device, characterized in that one or more layers are produced from solution, for example by spin-coating, or by any printing method, for example screen printing, flexographic printing, offset printing, LITI (light-induced thermal imaging, thermal transfer printing), inkjet printing or nozzle printing. For this purpose, soluble compounds are needed, which are obtained, for example, through suitable substitution.

In addition, hybrid methods are possible, in which, for example, one or more layers are applied from solution and one or more further layers are applied by vapor deposition.

Those skilled in the art are generally aware of these methods and are able to apply them without exercising inventive skill to organic electroluminescent devices comprising the compounds of the invention.

In particular, the electronic devices containing one or more deuterated compounds can be used in displays, as light sources in lighting applications and as light sources in medical and/or cosmetic applications (e.g. light therapy).

The deuterated compounds and the organic electroluminescent devices of the invention are notable for one or more of the following properties:
1. The compounds of the invention lead to long lifetimes.
2. The compounds of the invention lead to high efficiencies, especially to a high EQE.
3. The compounds of the invention lead to low operating voltages.

The invention is illustrated in detail by the examples which follow, without any intention of restricting it thereby. The person skilled in the art will be able to use the information given to execute the invention over the entire scope disclosed and to prepare further deuterated compounds without exercising inventive skill and to use them in electronic devices or to employ the process of the invention.

### EXAMPLES

The syntheses which follow, unless stated otherwise, are conducted under a protective gas atmosphere in dried solvents. The solvents and reagents can be purchased, for example, from Sigma-ALDRICH or ABCR. The respective figures in square brackets or the numbers quoted for individual compounds relate to the CAS numbers of the compounds known from the literature.

### Compounds Used

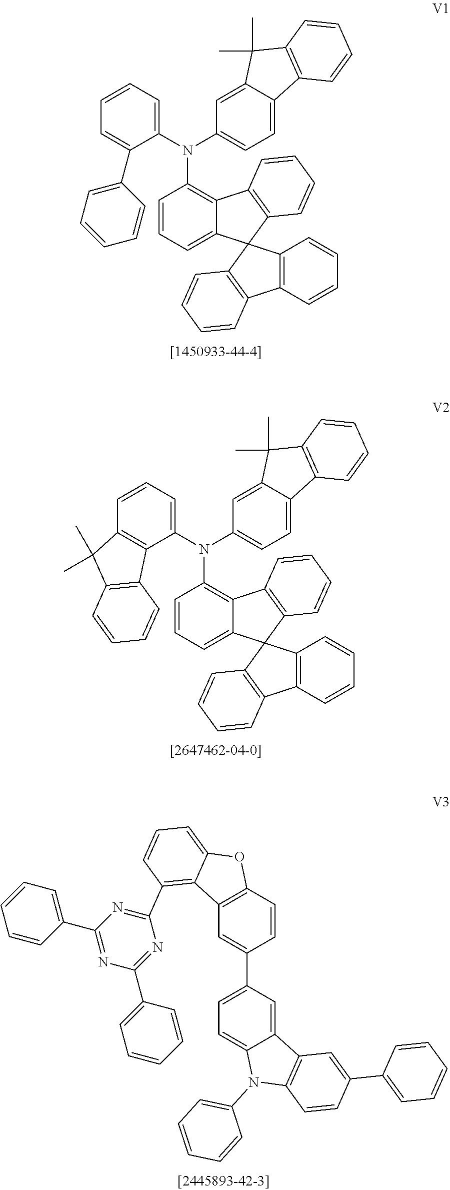 [SMILES: CC1(C)c2ccccc2-c2ccc(N(c3ccccc3-c3ccccc3)c3cccc4c3-c3ccccc3C43c4ccccc4-c4ccccc43)cc21 ; CC1(C)c2ccccc2-c2ccc(N(c3cccc4c3-c3ccccc3C4(C)C)c3cccc4c3-c3ccccc3C43c4ccccc4-c4ccccc43)cc21 ; c1ccc(-c2ccc3c(c2)c2cc(-c4ccc5oc6cccc(-c7nc(-c8ccccc8)nc(-c8ccccc8)n7)c6c5c4)ccc2n3-c2ccccc2)cc1]

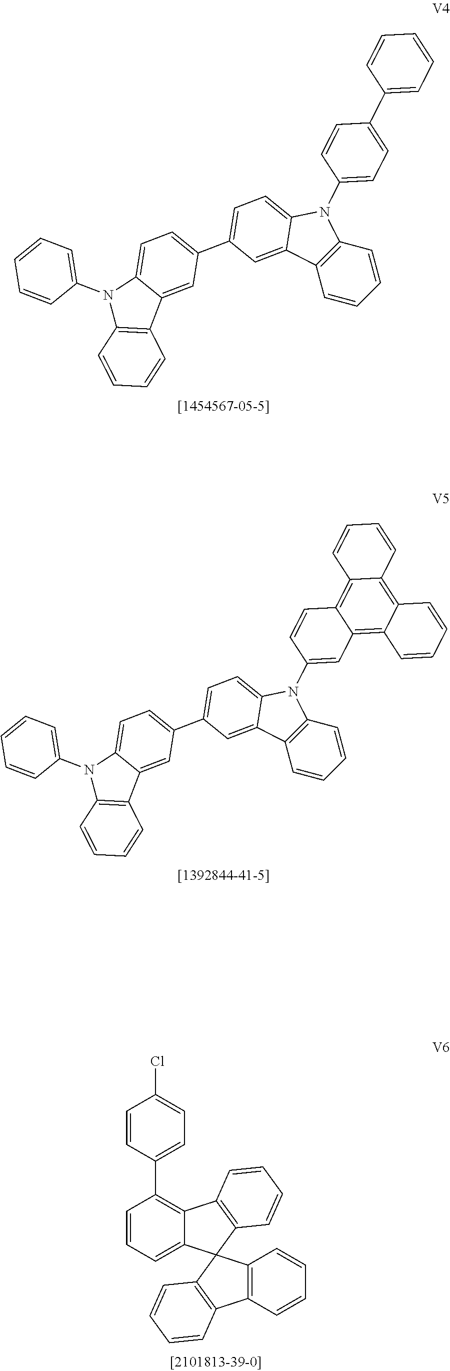 [SMILES: c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc5c6ccccc6c6ccccc6c5c4)ccc32)cc1 ; Clc1ccc(-c2cccc3c2-c2ccccc2C32c3ccccc3-c3ccccc32)cc1]

### Materials Used

Deuterium oxide—D₂O: Sigma Aldrich (≥99.9 atom % D)
Cyclohexane—Cy: MerckMillipore (≥99.5%)
Methylcyclopentane—Mcp: MerckMillipore (≥99.5%)
Decalin—Dec: cis/trans isomer mixture MerckMillipore (≥99.0%)
Palladium/charcoal—Pd/C; 5% by weight of Pd; Evonik Operations GmbH
Platinum/charcoal—Pt/C: 5% by weight of Pt; Evonik Operations GmbH
Triethylamine: MerckMillipore (≥99.0%)
Isopropanol—i-PrOH: MerckMillipore (≥99.5%)
2-Pentanol—2-P-OH: MerckMillipore (≥99.5%)
Potassium acetate—KOAc: Sigma Aldrich (≥99.9%)
Palladium(II) acetate—Pd(OAc)₂: Sigma Aldrich (99.98% trace metals basis)
Palladium(II) pivalate—Pd(OPiv)₂: Sigma Aldrich (97% assay)
Silver(I) acetate—Ag(OAc): Sigma Aldrich (99.99% trace metals basis)

### Drying of the Catalysts

The water-moist catalysts (Pd/C and Pt/C) were dried at 60° C. and 20 mbar in a vacuum drying cabinet for 3 d. Water content by Karl Fischer method about 1%.

### General Description of Procedure

### Method 1: Reaction Regime in an Autoclave Under Autogenous Pressure

A stirred autoclave is charged with the compound V, D₂O, a solvent (LM), a catalyst Kat.1, optionally a catalyst Kat.2, and optionally additives Add.1, Add.2, etc., inertized by two cycles of injection of nitrogen to 5 bar and decompression or by a single injection of nitrogen to 30 bar and decompression, and stirred with a pitched blade stirrer at 1000 rpm at the specified temperature T for the specified reaction time R. The exact batch volumes are shown below. The autoclave is allowed to cool down, the reaction mixture is removed and the catalyst is filtered off, and the cyclohexane phase is separated off. The catalyst is washed with THF and then extracted with hot THF until it no longer contains any product. The combined organic phases are concentrated to dryness under reduced pressure on a rotary evaporator (p about 20 mbar, T about 60° C.).

If, as described in the comparative examples, cis-/trans-decalin with a boiling point of 189-191° C. is used, it cannot be removed on a standard laboratory rotary evaporator; it has to be removed by a bridge under oil-pump vacuum (p about 10⁻² mbar, T about 80° C.).

### Method 2: Reaction Regime Under Atmospheric Pressure

Procedure as described above, but in a standard stirred apparatus (e.g. 2 I or 4 I four-neck flask, precision glass stirrer with perforated Teflon blade—350-4500 rpm, internal thermometer, reflux condenser, protective gas blanketing (nitrogen or argon)) under protective gas and reflux RF (about 72° C.) under atmospheric pressure. The entry RF in the reaction temperature column indicates that method 2 is used. Workup as described above.

### Determination of Purity and Level of Deuteration

Conversion (area % of the deuterated product) and by-products (total of the area percentages of all by-products) are determined by HPLC, Merck Hitachi D-7000, detection wavelength 254 nm, column: StarRP18e 250/4.5 5 μm, THF/ACN/H₂O mixtures.

The deuteration level M_{D} is determined by HPLC-MS, Agilent 1260 Infinity Il, ionization: APCI, column: Agilent Zorbax-C18 600 bar, 2.1×50 mm, 1.8 μm, THF/ACN/H₂O mixtures. For this purpose, an HPLC-MS chromatogram is created. This is used to ascertain the mass M+H⁺ of the most intense isotope combination. The ascertained M+H⁺ value is lowered by 1: M_{D}=M+H⁺−1.

The deuteration level M_{D} is calculated by the following formula:

$$Deuteration level = ( {M}_{D} - {M}_{H} ) / {N}_{H}$$

N_{H}: number of protons H
M_{H}: computed molecular mass (ChemDraw) of the undeuterated reactant
M_{D}: mass of the most intense isotope combination determined by HPLC-MS and lowered by 1

### Conversion of V1

### Reaction Mixture and Conditions

| | V1 | D₂O | | | | | | | |
|---|---|---|---|---|---|---|---|---|---|
| | [g] | [ml] | LM | Kat.1 | Kat.2 | Add.1 | Add.2 | T | R |
| Ex. | [mmol] | [mmol] | [ml] | [g] | [g] | [ml] | [ml] | [° C.] | [h] |
| V1A1 | 1.0 | 20.0 | Dec | Pt/C | — | 2-P—OH | — | 140 | 20 |
| | 1.5 | 1105.4 | 40.0 | 0.09 | | 4.0 | | | |
| V1A2 | 1.0 | 20.0 | Dec | Pt/C | — | i-PrOH | — | 140 | 20 |
| | 1.5 | 1105.4 | 40.0 | 0.09 | | 4.0 | | | |
| V1A3 | 1.0 | 20.0 | Cy | Pt/C | — | 2-P—OH | — | 140 | 20 |
| | 1.5 | 1105.4 | 40.0 | 0.09 | | 4.0 | | | |
| V1A4 | 1.0 | 20.0 | Cy | Pt/C | — | i-PrOH | — | 140 | 20 |
| | 1.5 | 1105.4 | 40.0 | 0.09 | | 4.0 | | | |
| V1B1 | 1.0 | 20.0 | Cy | Pt/C | — | — | — | 140 | 20 |
| | 1.5 | 1105.4 | 40.0 | 0.09 | | | | | |
| V1B2 | 1.0 | 20.0 | Cy | Pt/C | Pd/C | — | — | 140 | 20 |
| | 1.5 | 1105.4 | 40.0 | 0.08 | 0.02 | | | | |
| V1B3 | 1.0 | 20.0 | Cy | Pt/C | Pd/C | — | — | 140 | 20 |
| | 1.5 | 1105.4 | 40.0 | 0.40 | 0.10 | | | | |
| V1B4 | 1.0 | 20.0 | Dec | Pt/C | — | — | — | 180 | 20 |
| | 1.5 | 1105.4 | 20.0 | 0.40 | | | | | |

### Result

| | Conversion | By-products | Deuteration level |
|---|---|---|---|
| Ex. | [%] | [%] | [%] |
| V1A1 | 100.0 | 17.5 | 45 |
| V1A2 | 100.0 | 18.5 | 45 |
| V1A3 | 100.0 | 14.6 | 43 |
| V1A4 | 100.0 | 13.7 | 44 |
| V1B1 | 100.0 | 6.1 | 43 |
| V1B2 | 100.0 | 2.0 | 43 |
| V1B3 | 100.0 | 3.3 | 57 |
| V1B4 | 100.0 | 2.3 | 60 |

There is a distinct increase in by-products on addition of an alcohol. The use of a catalyst mixture reduces the amount of by-products again. An increase in the amount of catalyst improves deuteration.

### Conversion of V2

### Reaction Mixture and Conditions

| | V2 | D₂O | | | | | | | |
|---|---|---|---|---|---|---|---|---|---|
| | [g] | [ml] | LM | Kat.1 | Kat.2 | Add.1 | Add.2 | T | R |
| Ex. | [mmol] | [mmol] | [ml] | [g] | [g] | [ml] | [ml] | [° C.] | [h] |
| V2A1 | 1.2 | 6.0 | Dec | Pt/C | Pd/C | i-PrOH | — | 120 | 20 |
| | 1.7 | 331.6 | 48 | 0.48 | 0.12 | 4.8 | | | |
| V2B1 | 1.2 | 6.0 | Cy | Pt/C | — | — | — | 120 | 20 |
| | 1.7 | 331.6 | 48 | 0.48 | | | | | |
| V2B2 | 1.2 | 6.0 | Cy | Pt/C | Pd/C | — | — | 120 | 20 |
| | 1.7 | 331.6 | 48 | 0.48 | 0.12 | | | | |
| V2B3 | 1.2 | 6.0 | Cy | Pt/C | Pd/C | — | — | 120 | 20 |
| | 1.7 | 331.6 | 48 | 0.24 | 0.24 | | | | |
| V2B4 | 1.2 | 12.0 | Cy | Pt/C | Pd/C | — | — | 120 | 20 |
| | 1.7 | 663.3 | 48 | 0.24 | 0.24 | | | | |
| V2B5 | 2.0 | 20.0 | Cy | Pt/C | Pd/C | — | — | 120 | 20 |
| | 2.8 | 1105.4 | 40 | 0.80 | 0.20 | | | | |
| V2B6 | 50.0 | 500 | Cy | Pt/C | Pd/C | — | — | RF | 22 |
| | 70.0 | 27.6 mol | 2000 | 25 g | 25 g | | | | |
| V2B7 | 50.0 | 500 | Cy | Pt/C | Pd/C | — | — | RF | 46 |
| | 70.0 | 27.6 mol | 2000 | 25 g | 25 g | | | | |
| V2B8 | 50.0 | 500 | Dec | Pt/C | Pd/C | — | — | 160 | 20 |
| | 70.0 | 27.6 mol | 1000 | 25 g | 25 g | | | | |

### Result

| | Conversion | By-products | Deuteration level |
|---|---|---|---|
| Ex. | [%] | [%] | [%] |
| V2A1 | 100.0 | 5.9 | 30 |
| V2B1 | 100.0 | 3.1 | 28 |
| V2B2 | 100.0 | 0.4 | 30 |
| V2B3 | 100.0 | 0.1 | 28 |
| V2B4 | 100.0 | 0.2 | 30 |
| V2B5 | 100.0 | 0.4 | 35 |
| V2B6 | 100.0 | 0.1 | 32 |
| V2B7 | 100.0 | 0.2 | 38 |
| V2B8 | 100.0 | 0.3 | 44 |

The process of the invention, even under reflux conditions, leads to good results, especially to low by-products. The addition of an alcohol increases the amount of by-products again.

### Conversion of V3

### Reaction Mixture and Conditions

| | V3 | D₂O | | | | | | | |
|---|---|---|---|---|---|---|---|---|---|
| | [g] | [ml] | LM | Kat.1 | Kat.2 | Add.1 | Add.2 | T | R |
| Ex. | [mmol] | [mmol] | [ml] | [g] | [g] | [g] | [ml] | [° C.] | [h] |
| V3A1 | 1.0 | 20 | Dec | Pt/C | Pd/C | i-PrOH | — | 120 | 20 |
| | 1.6 | 1105.4 | 40 | 0.40 | 0.10 | 4.0 | | | |
| V3B1 | 1.0 | 20 | Cy | Pt/C* | Pd/C* | — | — | 120 | 20 |
| | 1.6 | 1105.4 | 40 | 0.40 | 0.10 | | | | |
| V3B2 | 1.0 | 20 | Mcp | Pt/C | Pd/C | — | — | 120 | 20 |
| | 1.6 | 1105.4 | 40 | 0.40 | 0.10 | | | | |
| V3B3 | 1.0 | 20 | — | Pt/C | Pd/C | — | — | 120 | 20 |
| | 1.6 | 1105.4 | | 0.40 | 0.10 | | | | |
| V3B4 | 1.0 | 20 | Cy | Pt/C | Pd/C | — | — | 120 | 20 |
| | 1.6 | 1105.4 | 40 | 0.40 | 0.10 | | | | |
| V3B5 | 1.0 | 20 | Cy | Pt/C | Pd/C | — | — | 120 | 20 |
| | 1.6 | 1105.4 | 40 | 0.20 | 0.10 | | | | |
| V3B6 | 1.0 | 20 | Cy | Pt/C | Pd/C | — | — | 120 | 96 |
| | 1.6 | 1105.4 | 40 | 0.40 | 0.20 | | | | |
| V3B7 | 1.0 | 20 | Dec | Pt/C | Pd/C | — | — | 180 | 16 |
| | 1.6 | 1105.4 | 20 | 0.40 | 0.20 | | | | |

*catalyst was not dried

### Result

| | Conversion | By-products | Deuteration level |
|---|---|---|---|
| Ex. | [%] | [%] | [%] |
| V3A1 | 100 | 5.1 | 71 |
| V3B1 | 0.0 | 0.1 | 0 |
| V3B2 | 0.0 | 0.2 | 0 |
| V3B3 | 0.0 | 0.0 | 0 |
| V3B4 | 100.0 | 1.0 | 73 |
| V3B5 | 100.0 | 0.2 | 59 |
| V3B6 | 100.0 | 0.8 | 81 |
| V3B7 | 100.0 | 1.1 | 85 |

The use of undried catalyst (V3B1) leads to no conversion. Nor does the use of methylcyclopropane lead to any product. The extension of the reaction time increases by-products, but also the level of deuteration.

### Conversion of V4

### Reaction Mixture and Conditions

| | V4 | D₂O | | | | | | | |
|---|---|---|---|---|---|---|---|---|---|
| | [g] | [ml] | LM | Kat.1 | Kat.2 | Add.1 | Add.2 | T | R |
| Ex. | [mmol] | [mmol] | [ml] | [g] | [g] | [μl] | [mg] | [° C.] | [h] |
| V4A1 | 1.2 | 7 | Dec | Pt/C | Pd/C | i-PrOH | — | 120 | 20 |
| | 2.1 | 386.9 | 48 | 0.48 | 0.12 | 4.0 | | | |
| V4B1 | 1.2 | 7 | Cy | Pt/C | Pd/C | — | — | 100 | 20 |
| | 2.1 | 386.9 | 48 | 0.48 | 0.12 | | | | |
| V4B2 | 1.2 | 7 | Cy | Pt/C | Pd/C | — | — | 120 | 20 |
| | 2.1 | 386.9 | 48 | 0.48 | 0.12 | | | | |
| V4B3 | 1.2 | 7 | Cy | Pt/C | Pd/C | — | — | 140 | 20 |
| | 2.1 | 386.9 | 48 | 0.48 | 0.12 | | | | |
| V4B4 | 1.2 | 7 | Cy | Pt/C | Pd/C | NEt₃ | — | 120 | 20 |
| | 2.1 | 386.9 | 48 | 0.48 | 0.12 | 15 | | | |
| V4B5 | 1.2 | 7 | Cy | Pt/C | Pd/C | NEt₃ | — | 120 | 20 |
| | 2.1 | 386.9 | 48 | 0.48 | 0.12 | 30 | | | |
| V4B6 | 1.2 | 7 | Cy | Pt/C | Pd/C | NEt₃ | — | 120 | 20 |
| | 2.1 | 386.9 | 48 | 0.48 | 0.12 | 59 | | | |
| V4B7 | 1.2 | 7 | Cy | Pt/C | Pd/C | — | Pd(OAc)₂ | 120 | 20 |
| | 2.1 | 386.9 | 48 | 0.48 | 0.12 | | 40 | | |
| V4B8 | 1.2 | 7 | Cy | Pt/C | — | — | Pd(OAc)₂ | 120 | 20 |
| | 2.1 | 386.9 | 48 | 0.48 | | | 80 | | |
| V4B9 | 1.2 | 7 | Cy | Pt/C | Pd/C | — | Ag(OAc) | 120 | 20 |
| | 2.1 | 386.9 | 48 | 0.48 | 0.12 | | 36 | | |
| V4B10 | 60 | 350 | Cy | Pt/C | Pd/C | — | — | 120 | 20 |
| | 107 | 19.3 mol | 3000 | 24.0 | 6.0 | | | | |
| V4B11 | 1.2 | 7 | Cy | Pt/C | — | — | — | 120 | 20 |
| | 2.1 | 386.9 | 48 | 0.48 | | | | | |
| V4B12 | 1.2 | 7 | Cy | Pt/C | Pd/C | — | Pd(OPiv)₂ | 120 | 20 |
| | 2.1 | 386.9 | 48 | 0.48 | 0.12 | | 40 | | |
| V4B13 | 60 | 350 | Dec | Pt/C | Pd/C | — | — | 160 | 12 |
| | 107 | 19.3 mol | 1000 | 24.0 | 6.0 | | | | |

### Result

| | Conversion | By-products | Deuteration level |
|---|---|---|---|
| Ex. | [%] | [%] | [%] |
| V4A1 | 100 | 27.6 | 70 |
| V4B1 | 36.4 | 0.0 | 49 |
| V4B2 | 94.0 | 5.7 | 60 |
| V4B3 | 100.0 | 40.0 | 74 |
| V4B4 | 84.9 | 4.6 | 56 |
| V4B5 | 85.6 | 1.1 | 49 |
| V4B6 | 77.3 | 1.4 | 45 |
| V4B7 | 98.9 | 1.2 | 70 |
| V4B8 | 78.3 | 0.9 | 77 |
| V4B9 | 100.0 | 3.8 | 67 |
| V4B10 | 99.6 | 8.7 | 67 |
| V4B11 | 99.9 | 12.8 | 67 |
| V4B12 | 79.8 | 0.5 | 74 |
| V4B13 | 100.0 | 1.9 | 71 |

The addition of the additive lowers by-products and, in the case of the Pd salts, also increases the level of deuteration. This can increase conversion.

### Conversion of V5

### Reaction Mixture and Conditions

| | V5 | D₂O | | | | | | | |
|---|---|---|---|---|---|---|---|---|---|
| | [g] | [ml] | LM | Kat.1 | Kat.2 | Add.1 | Add.2 | T | R |
| Ex. | [mmol] | [mmol] | [ml] | [g] | [g] | [μl] | [mg] | [° C.] | [h] |
| V5A1 | 1.2 | 7 | Dec | Pt/C | Pd/C | i- | — | 140 | 20 |
| | 1.9 | 386.9 | 48 | 0.48 | 0.12 | PrOH | | | |
| | | | | | | 4.0 ml | | | |
| V5B1 | 1.2 | 7 | Cy | Pt/C | Pd/C | — | — | 120 | 20 |
| | 1.9 | 386.9 | 48 | 0.48 | 0.12 | | | | |
| V5B2 | 1.2 | 7 | Cy | Pt/C | Pd/C | — | — | 140 | 20 |
| | 1.9 | 386.9 | 48 | 0.48 | 0.12 | | | | |
| V5B3 | 1.2 | 7 | Cy | Pt/C | Pd/C | — | Pd(OAc)₂ | 140 | 20 |
| | 1.9 | 386.9 | 48 | 0.48 | 0.12 | | 43 | | |
| V5B4 | 1.2 | 7 | Cy | Pt/C | Pd/C | NEt₃ | — | 140 | 20 |
| | 1.9 | 386.9 | 48 | 0.48 | 0.12 | 26 | | | |

### Result

| | Conversion | By-products | Deuteration level |
|---|---|---|---|
| Ex. | [%] | [%] | [%] |
| V5A1 | 100.0 | 49.7 | 62 |
| V5B1 | 46.4 | 0.6 | 48 |
| V5B2 | 77.5 | 11.5 | 58 |
| V5B3 | 96.9 | 8.1 | 88 |
| V5B4 | 83.3 | 5.5 | 49 |

The additives again lead to an increase in deuteration level and/or to reduction of by-products.

### Conversion of V6

### Reaction Mixture and Conditions

| | V6 | D₂O | | | | | | | |
|---|---|---|---|---|---|---|---|---|---|
| | [g] | [ml] | LM | Kat.1 | Kat.2 | Add.1 | Add.2 | T | R |
| Ex. | [mmol] | [mmol] | [ml] | [g] | [g] | [ml] | [mg] | [° C.] | [h] |
| V6B1 | 1.0 | 10.0 | Cy | Pt/C | — | — | — | 100 | 20 |
| | 2.3 | 552.7 | 25 | 0.50 | | | | | |

### Result

| | Conversion | By-products | Deuteration level |
|---|---|---|---|
| Ex. | [%] | [%] | [%] |
| V6B1 | 100.0 | 1.0 | 24 |

The example shows that chlorine groups are tolerated as well.

1. A process for preparing a deuterated organic compound, comprising the steps of:
a) providing at least one heterogeneous metal catalyst, wherein the providing comprises drying of the metal catalyst;
b) preparing a liquid composition comprising at least one organic compound, the at least one heterogeneous metal catalyst, at least one deuterium source, and at least one aliphatic hydrocarbon as solvent, and
c) heating the composition to deuterate the organic compound.

2. The process as claimed in claim 1, wherein the heterogeneous metal catalyst is selected from the group comprising platinum, palladium, rhodium, ruthenium, nickel, cobalt, oxides thereof and combinations thereof.

3. The process as claimed in claim 1, wherein the drying is conducted down to a water content of below 5% by weight as measured by the Karl Fischer test.

4. The process as claimed in claim 1, wherein the organic compound is selected from the group consisting of an aromatic compound, a heteroaromatic compound, and an organometallic compound.

5. The process as claimed in claim 1, wherein the deuterium source is selected from the group consisting of heavy water, D₂O, d6-benzene, and d8-toluene.

6. The process as claimed in claim 1, wherein the solvent is an aliphatic solvent having a boiling point exceeding 75° C.

7. The process as claimed in claim 1, wherein the composition does not comprise any aliphatic alcohols.

8. The process as claimed in claim 1, wherein a ratio of hydrogen atoms in the organic compound to deuterium in the deuterium source is at least 1:1.5.

9. The process as claimed in claim 1, wherein a ratio of deuterium source to solvent, measured by volume, is 2:1 to 1:50.

10. The process as claimed in claim 1, wherein step c) involves heating to a temperature of at least 40° C.

11. The process as claimed in claim 10, wherein step c) involves heating to a temperature of up to 200° C.

12. The process as claimed in claim 1, wherein step c) is conducted with or without equalization of pressure with the environment.

13. The process as claimed in claim 1, wherein the composition comprises at least one additive selected from the group consisting of alkylamines, metal salts and metal oxides.

14. The process as claimed in claim 13, wherein the metal salts and metal oxides are selected from the group consisting of salts and oxides of palladium, platinum, rhodium, ruthenium, silver, gold, copper, nickel and cobalt.

15. The process as claimed in claim 1, wherein the solvent is cyclohexane.
